(19) United States
(12) Patent Application Publication
(10) Pub. No.: US 2005/0107465 A1
(43) Pub. Date: May 19, 2005

(54) Composition for treating inflammatory bowel disease

(76) Inventors: Andreas M. Papas, Kingsport, TN (US); Konstantinos A. Papas, Jonesborough, TN (US); J. Steven Alexander, Shreveport, LA (US)

Correspondence Address: SUSAN F. JOHNSTON;SUSAN F. JOHNSTON, ATTORNEY AT LAW, P.O. BOX 4449, JOHNSON CITY, TN, 37602-4449, US

(21) Appl. No.: 10/872,249

(22) Filed: Jun. 18, 2004

Related U.S. Application Data
(60) Provisional application No. 60/507,825, filed on Oct. 1, 2003.

(51) Int. Cl. A61K 31/355

(52) U.S. Cl. 514/458

(57) ABSTRACT

A method for reducing inflammation in inflammatory bowel disease is disclosed. The method comprises reducing the expression of endothelial cell adhesion molecules in the vasculature of the bowel wall by administering a therapeutic dosage of an effective amount of a blend of vitamin E TPGS and gamma-tocopherol. Gamma-tocopherol is also useful when used alone in the invention. The reduction in endothelial cell adhesion molecules is due to the deactivation of endothelial NFκ-B by the composition.

### CROSS REFERENCES TO RELATED APPLICATIONS

This application claims the benefit of U.S. Provisional Application Serial No. 60/507,825 filed Oct. 1, 2003.

### BACKGROUND OF THE INVENTION

Crohn's disease and ulcerative colitis are the two primary disorders that comprise the category of Inflammatory Bowel Diseases (IBD). Crohn's disease causes full thickness bowel wall inflammation and may involve any part of the digestive tract, from mouth to anus, while ulcerative colitis primarily targets the colon and results in more superficial bowel wall involvement. Absorption of dietary nutrients is typically impaired due to the inflammation of the intestine, diarrhea, cholestasis and medications. There is a significant increase in oxidative stress in IBD patients, partly due to malabsorption of dietary antioxidants. Production of endogenous antioxidants may also be impaired due to poor availability of essential cofactors such as zinc and selenium. The other cause of oxidative stress in IBD is the formation of reactive oxygen species and reactive nitrogen species as a result of the acute inflammatory process characteristic of IBD.

The intestinal mucosal immune system is constantly stimulated by lumenal contents and bacteria. In the normal system, pro-inflammatory and anti-inflammatory cells and other molecules are carefully regulated to promote a normal host mucosal defense capability without destruction of intestinal tissue. In IBD, immunoregulatory mechanisms are disrupted, resulting in an exaggerated immune response. Researchers believe that IBD is somehow initiated by macrophage activation, probably in response to a bacteria, virus or other foreign molecule. While the etiology of IBD is still unknown, the pathophysiology of this disease state is now partly known.

As shown in FIG. 1, the exaggerated immune response in IBD results in the release of large amounts of inflammatory cytokines and chemokines, including TNF-α. This cascade of inflammatory mediators causes the expression of endothelial cell adhesion molecules (ECAM) in the bowel area to which blood stream leukocytes adhere. Once adhered to the adhesion cell molecules, leukocytes are extravasated from the blood vessel into the bowel wall tissue. Since the bowel wall tissue is very vascularized, extravasated leukocytes can be present all across the thickness of the bowel wall. This over-presence of leukocytes in the bowel wall tissue results in the chronic inflammation that is characteristic of IBD. More specifically, the inability of IBD patients to effectively regulate the activation of tissue macrophages or the invasion of inflamed tissue with more phagocytic leukocytes results in the production of deleterious reactive oxygen species (ROS) and reactive nitrogen species (RNS) that cause cellular stress and apoptosis.

The most recent therapies for IBD have attempted to prevent extravasation of leukocytes into the bowel wall tissue. As stated above, the extravasation of leukocytes results from the expression of ECAM that cause adhesion of leukocytes to the endothelial wall of blood vessels throughout the bowel. The expression of ECAM, such as intercellular cell adhesion molecule-1 (ICAM-1), vascular cell adhesion molecule-1 (VCAM-1), and mucosal addressin cell adhesion molecule-1 (MAdCAM-1), in the vasculature of the bowel is increased during IBD. To this end, the most recent therapies for IBD have attempted to block TNF-α mediated ECAM expression with anti-TNF-α agents such as REMICADE (trade name for inflixamab). However, these therapies have significant and severe system side-effects. Sasaki, et al found that melatonin may exert therapeutic activity in IBD through its ability to inhibit NF-κB dependent induction of MAdCAM-1. (Makoto Sasaki, et al. BMC Gastroenterology 2002. 2:9) However, the amount of melatonin required to inhibit the induction of MAdCAM-1 is deleteriously high. Sasaki et al reported that ECAM over-expression in IBD is caused by the activation of the NF-κB transcriptional system when tumor necrosis factor alpha (TNF-α) is released by macrophages in the liver.

It is an object of the present invention to provide a new therapy for reducing the exaggerated inflammatory response during IBD. It is another object of the present invention to prevent or reduce the expression of ECAM as part of the immunoregulatory response in IBD using a therapy that does not have deleterious side effects. It is a further object of the present invention to provide a therapy using a composition that does not require use of steroids, monoclonal antibodies, antibiotics, or immunosuppressants such as methotraxate, cyclosporine,_azathioprine, and mercaptopurine.

### BRIEF SUMMARY OF THE INVENTION

The present invention is a method for treating a patient having inflammatory bowel disease and the compound and composition used in the therapeutic method. The method comprises reducing the expression of endothelial cell adhesion molecules in the vasculature of the bowel wall, including mucosal addressin cell adhesion molecule-1 (MAdCAM-1), by administering a therapeutic dosage of an effective amount of a gamma-homolog of vitamin E selected from the group consisting of gamma-tocopherol, esters of gamma-tocopherol, gamma-tocotrienol, esters of gamma-tocotrienol, and a combination thereof. In the preferred embodiment of the invention, the gamma-homolog of vitamin E is blended with vitamin E TPGS to provide an especially synergistic composition in the treatment of IBD.

### BRIEF DESCRIPTION OF THE DRAWINGS

FIG. 1 is a flow diagram of a portion of the exaggerated immune response in IBD.

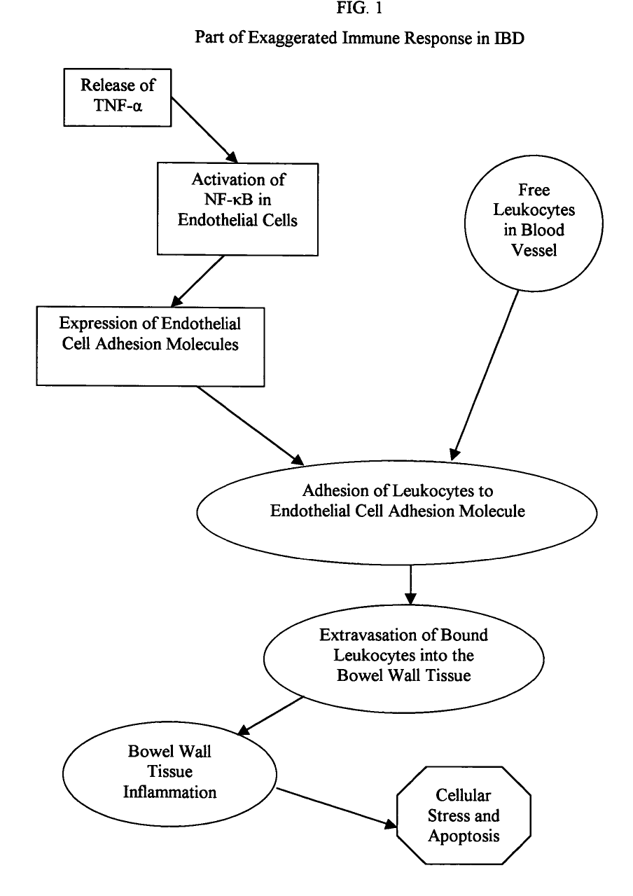

FIG. 2 shows electrophoretic scan density data of the concentration of TNF-α in endothelial cells that had been pre-treated with TNF-α after undergoing therapy with gamma-tocopherol alone, therapy with vitamin E TPGS alone, and therapy with a combination of gamma-tocopherol and vitamin E TPGS. FIG. 2 further illustrates the correlation between TNF-α concentration and MAdCAM-1 expression.

### DETAILED DESCRIPTION

The inventors have discovered that the gamma-tocopherol form of vitamin E demonstrates significant therapeutic effectiveness in reducing the expression of MAdCAM-1 in murine colon endothelial cells. Thus, an IBD patient treated in vivo with a therapeutically effective dose of γ-tocopherol would likewise demonstrate a reduction in MAdCAM-1 expression. This finding is surprising because prior use of gamma-tocopherol in IBD has been limited to its use as a general antioxidant for reducing the number of reactive oxygen and nitrogen species formed in the IBD response and as a formulation co-agent for eliminating reactive species produced by an active therapeutic agent. Examples include Shapiro (U.S. Pat. No. 6,444,221) and Guivarc'h et al (U.S. Ser. No. 2003/0013693 A1).

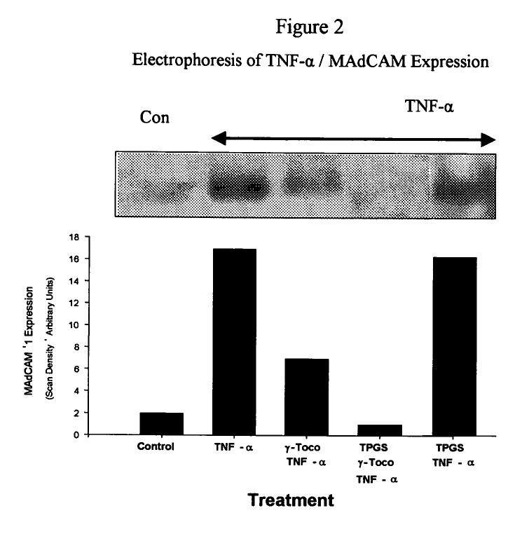

As shown in FIG. 2, the inventors have found that gamma-tocopherol interferes with the ability of TNF-α to induce the expression of MAdCAM possibly due to inhibition of NF-κB. The effectiveness of gamma-tocopherol in reducing MAdCAM-1 expression in IBD is even further surprising due to the fact that the delivery of more prolifically used anti-oxidants such as alpha-tocopherol, coenzyme Q₁₀, and vitamin E TGPS, showed a relatively minor effect in reducing the expression of MAdCAM-1.

The active agent of the present invention is not limited to gamma-tocopherol alone. The esters of gamma-tocopherol are also included. Further, gamma-tocotrienol and the esters thereof are also included as suitable active agents in the present invention since their biological activity so closely mirrors that of gamma-tocopherol. Examples of suitable esters of gamma-tocopherol and gamma-tocotrienol include the succinate esters, polyethylene glycol succinate esters, acetates, nicotinates, and phosphates, and combinations thereof. Thus, the composition is referred to hereinafter as a “gamma-T” composition for convenience.

In the preferred embodiment of the present invention, the IBD patient is treated with a blend of gamma-T and a solubilizing amount of vitamin E TPGS. Vitamin E TPGS is a water-soluble form of natural-source vitamin E prepared by esterifying d-alpha-tocopheryl acid succinate with polyethylene glycol 1000. Vitamin E TPGS is a well known compound having a chemical formula of C₃₃O₅H₅₄(CH₂CH₂O)ₙ, where “n” represents the number of polyethylene oxide moieties attached to the acid group of crystalline d-alpha tocopheryl acid succinate. As further shown in FIG. 2, while in vitro treatment of IBD endothelial cells with gamma-T alone was found to significantly decrease the expression of MAdCAM-1 and treatment with vitamin E TPGS alone was found to provide a relatively small effect, therapy using a blend of gamma-T and vitamin E TPGS was found to fully suppress the cytokine mediated responses. Thus, the combination of gamma-T with vitamin E TPGS has a particularly synergistic relationship in IBD which provides an unexpectedly high degree of MAdCAM-1 suppression.

It is a poignant aspect of the present invention that the gamma-T or gamma-T/TPGS blend action is not dependent upon the presence of any other bioactive compound for interfering with cytokine mediated expression of MAdCAM in IBD. Thus, the present gamma-T therapy is effective in the absence of an antibiotic, steroid, monoclonal antibody or other anti-inflammatory, or immune suppressing agent.

The effective in vivo dosage of gamma-T for administration in the present invention is a daily oral dosage of about 500 to 15,000 mg gamma-T, preferably between 1,500 to about 5,000 mg gamma-T should be effective in reducing MAdCAM-1 expression. At such dosage levels, it is especially beneficial to employ the preferred embodiment of a blend of gamma-T and vitamin E TPGS since the combination is so more effective than gamma-T alone, thus requiring a smaller amount of total vitamin E. Thus, the therapy is beneficial in treating both chronic and acute IBD conditions.

In the present invention, the gamma-T composition may be administered by any effective route including orally, intravenously, or rectally. Oral dosage forms include aqueous emulsions, gelatin capsules, and tablets.

The gamma-T composition is formed by melt blending gamma-T with a suitable carrier or solubilizer via commonly known methods. Vitamin E TPGS is the preferred solubilizer in this invention due to the synergistic therapeutic effect. However, other well known pharmaceutically acceptable solubilizers can be used as carrier for gamma-T in the present invention. Melt blending techniques are well known in the industry.

Other compounds may be incorporated into the gamma-T composition to provide dietary supplement benefit, antioxidant benefit, anti-inflammatory benefit, and the like. Examples of other compounds useful in the present gamma-T composition include coenzyme Q₁₀, other natural and synthetic homologs of vitamin E and esters thereof, vitamin C, zinc, and selenium. Monoclonal antibody compounds, antibiotics, immunosuppressants, and steroidal compounds can also be combined with the gamma-T and gamma-T/TPGS composition. Up to about 25 weight percent of the composition of the present invention can be such other therapeutically active compounds. However, the predominant component of the composition is gamma-T and vitamin E TPGS.

The composition of the present invention comprises gamma-T and a solubilizing amount of vitamin E TPGS. The concentration of the blend is between about 10 to 70 weight percent gamma-T, from 30 to about 90 weight percent vitamin E TPGS, and from 0 to about 25 weight percent other therapeutically active compounds. The preferred embodiment includes between about 30 to about 60 weight percent gamma-T and between about 40 to about 70 weight percent vitamin E TPGS, with a presence of no more than 15 weight percent other active compounds being most preferred. It should be understood that the composition of the present invention can be combined with other non-bioactive ingredients in formulating a dosage form. Formulation components such as granulation aids, binders, flow agents, colorants, preservatives and fillers are examples of such non-active formulation ingredients.

While the present invention has been primarily directed to the treatment of IBD, other chronic and acute disease states and conditions brought about by the immunoregulatory production of TNF-α or inhibition of NF-κB would also be treatable, at least to some extent, with the present gamma-T composition.

This invention is further illustrated by the following examples of preferred embodiments thereof, although it will be understood that these examples are included merely for purposes of illustration and are not intended to limit the scope of the invention unless otherwise specifically indicated.

### EXAMPLE

Cell Culture:

A line of murine colon microvessel endothelial cells was developed for use as an in vitro model of colon inflammatory adhesion molecule expression in IBD. Immortomouse mice were used as tissue sources for these studies. Colon segments were sterilely removed and treated with collagenase (0.2%, 45 min) and cultured in d-valine MEM with IFN-g (10 U/ml) with 2% FCS and non-essential amino acids and vitamins. These cells were cultured on gelatin and were maintained at 37 C. for 24 hours prior to exposure to treatment protocols. In treatments, cells cultured on 24 well plates were incubated in either medium, or medium containing antioxidant or antioxidant admixture ± vitamin E TPGS for 24-48 hour prior to the administration of inflammatory cytokine (TNF-α, 20 ng/ml). At 24 hours combined treatments, monolayers were evaluated for MAdCAM-1 by standard western blotting.

Western Blot Assay for MAdCAM-1 Expression:

EC cells developed as above were cultured in 2% fetal calf serum, with 50 U/ml interferon-gamma, d-valine supplemented MEM, 1% penicillin/streptomycin and 1 % non-essential amino acids using tissue culture were pre-coated with 1 % gelatin. Adhesion molecule expression was measured in these cells by switching cells to medium without interferon, culturing for 48 hours and then pre-treating cells with test agents for 24 hours prior to exposure to TNF-α for an additional 24 hours. MAdCAM-1 expression was measured using standard western blotting procedures described above. One hundred μg of protein was loaded per lane in non-denaturing sample buffer (w/o mercaptoethanol) and electrophoresed, and subsequently transferred to nitrocellulose. After blocking (5% milk, 2 h), blots were incubated in MAdCAM-1 antibody (10 ug/ml) for 12 hours and reacted by enhanced chemiluminescence (ECL). Gels were analyzed by densitometric analysis by scanning x-ray replicas of the exposed blots.

NF-κB Activation:

Electrophoretic mobility shift assay (EMSA): NF-κB binding activity was performed in a 15 μl binding mixture containing 1× binding buffer (50 ug/ml of double-stranded poly (dl-dC), 10 mM Tris-HCl (pH 7.5), 50 mM NaCl, 0.5 mM EDTA, 0.5 mM DTT, 1 mM MgCl2, and 10% glycerol); 15 μg of nuclear proteins; and 35 fmol (50,000 CPM, Cherenkov counting) of double-stranded NF-κB consensus oligonucleotide (5′-AGT TGA GGG GAC TTT CCC AGG C-3′) end-labeled with ['g-32P] ATP (3,000 Ci/mmol at 10 mCi/ml; Amersham, Arlington Heights, Ill.) using T4 polynucleotide kinase (Promega). The relative binding reaction mixture was incubated at room temperature for 20 minutes and analyzed by electro-phoresis on 5% non-denaturing polyacrylamide gels. After electrophoresis, the gels were dried using a gel drier and exposed to Kodak X-ray film (−70° C.). The relative binding bands were analyzed by scanning densitometry on a Bio-Image Analysis System (Millipore Imaging System, Ann Arbor, Mich.).

### Example 1

A murine colon microvessel endothelial cell line (EC monolayer) was developed as described above. Cytokine induced MAdCAM-1 expression in these cells was determined via Western Blot assay described above. An initial experiment evaluated the ability of gamma-tocopherol alone or formulated with vitamin E TPGS to block cytokine induction of MAdCAM-1 in this cell line is shown in FIG. 2. Control (untreated) EC monolayers did not basally express MAdCAM-1, while stimulation with TNF-α (20 ng/ml) for 24 hours produced a robust increase in its expression. By comparison, gamma-tocopherol therapy produced a significant decrease in MAdCAM-1 expression (reduced by at least 50%), vitamin E TPGS therapy produced only a slight decrease in MAdCAM-1 expression, while therapy with a blend of gamma-tocopherol with vitamin E TPGS resulted in almost complete inhibition. Therapy with alpha-tocopherol produced a decrease in MAdCAM-1 expression that was significantly less effective than gamma-tocopherol alone. However, the distinct synergistic improvement gained in the combination of gamma-tocopherol and vitamin E TPGS was not displayed with any other combination of vitamin E TPGS or gamma-tocopherol. Coenzyme Q₁₀ therapy was less beneficial than alpha-tocopherol therapy.

These data clearly indicated that the formulation of gamma-tocopherol with vitamin E TPGS provides a superior protection against inflammation in IBD.

1. A method for treating a patient having inflammatory bowel disease, comprising reducing the expression of endothelial cell adhesion molecules in the vasculature of the bowel wall, including mucosal addressin cell adhesion molecule-1 (MAdCAM-1), by administering a therapeutic dosage of an effective amount of a gamma-homolog of vitamin E selected from the group consisting of gamma-tocopherol, esters of gamma-tocopherol, gamma-tocotrienol, esters of gamma-tocotrienol, and a combination thereof.

2. The method according to claim 1 wherein said gamma-homolog of vitamin E is gamma-tocopherol or an ester thereof.

3. The method according to claim 1 wherein the effective amount is between about 1,500 to about 5,000 mg gamma-homolog of vitamin E per day.

4. The method according to claim 1 wherein the therapeutic dosage is administered orally.

5. The method according to claim 1 wherein the therapeutic dosage is administered intravenously.

6. The method according to claim 1 wherein the therapeutic dosage is administered rectally.

7. A method for treating a patient having inflammatory bowel disease, comprising reducing the expression of endothelial cell adhesion molecules in the vasculature of the bowel wall, including mucosal addressin cell adhesion molecule-1 (MAdCAM-1), by administering a therapeutic dosage of an effective amount of a composition comprising from about 30 to about 90 weight percent vitamin E TPGS and from about 10 to about 70 weight percent of a gamma-homolog of vitamin E selected from the group consisting of gamma-tocopherol, esters of gamma-tocopherol, gamma-tocotrienol, esters of gamma-tocotrienol, and a combination thereof.

8. The method according to claim 7 wherein said composition has a concentration of from about 40 to about 70 weight percent vitamin E TPGS and from about 30 to about 60 weight percent gamma-homolog of vitamin E.

9. The method according to claim 7 wherein said gamma-homolog of vitamin E is gamma-tocopherol or an ester thereof.

10. The method according to claim 7 wherein the effective amount comprises between about 1,500 to about 5,000 mg gamma-homolog of vitamin E per day.

11. The method according to claim 7 wherein the therapeutic dosage is administered orally.

12. The method according to claim 7 wherein the therapeutic dosage is administered intravenously.

13. The method according to claim 7 wherein the therapeutic dosage is administered rectally.

14. A method for treating inflammatory bowel disease comprising:
administering a therapeutic dosage of a composition comprising from about 10 to about 70 weight percent of a gamma-homolog of vitamin E selected from the group consisting of gamma-tocopherol, esters of gamma-tocopherol, gamma-tocotrienol, esters of gamma-tocotrienol, and a combination thereof, from about 30 to about 90 weight percent vitamin E TPGS, and from 0 to about 25 weight percent other therapeutically active compounds wherein the summation of the weight percentages equals a total of 100 weight percent, wherein the therapeutic dosage is administered in an amount effective for reducing the expression of mucosal addressin cell adhesion molecule-1 (MAdCAM-1) in the vasculature of the bowel wall.

15. The method according to claim 14 wherein said other therapeutically active compounds in the composition are non-antibiotic, non-steroidal, non-immunosuppressant, non-monoclonal antibody compounds.

16. The method according to claim 14 wherein the effective amount comprises a concentration of about 1,500 to about 5,000 mg gamma-homolog of vitamin E per day.

17. A method for reducing inflammation in a patient having an exaggerated immune response comprising reducing the expression of endothelial cell adhesion molecules, including mucosal addressin cell adhesion molecule-1 (MAdCAM-1), by administering a therapeutic dosage of an effective amount of a composition comprising from about 30 to about 90 weight percent vitamin E TPGS and from about 10 to about 70 weight percent of a gamma-homolog of vitamin E selected from the group consisting of gamma-tocopherol, esters of gamma-tocopherol, gamma-tocotrienol, esters of gamma-tocotrienol, and a combination thereof.

18. The method according to claim 17 wherein the effective amount comprises between about 1,500 to about 5,000 mg gamma-homolog of vitamin E per day.

19. A composition for treating inflammatory bowel disease comprising:
from about 30 to about 90 weight percent vitamin E TPGS and from about 10 to about 70 weight percent of a gamma-homolog of vitamin E selected from the group consisting of gamma-tocopherol, esters of gamma-tocopherol, gamma-tocotrienol, esters of gamma-tocotrienol, and a combination thereof.

20. The composition according to claim 19 wherein the concentration of said vitamin E TPGS is from about 40 to about 70 weight percent of said composition and the concentration of said gamma-homolog of vitamin E is from about 30 to about 60 weight percent of said composition.

21. The composition according to claim 19 wherein said esters of gamma-tocopherol and said esters of gamma-tocotrienol include the succinate, polyethylene glycol succinate, acetate, nicotinate, and phosphate esters.

22. An oral medicament formed from the composition according to claim 19.

23. A composition for treating inflammatory bowel disease comprising:
a) about 10 to about 70 weight percent of a gamma-homolog of vitamin E selected from the group consisting of gamma-tocopherol, esters of gamma-tocopherol, gamma-tocotrienol, esters of gamma-tocotrienol, and a combination thereof;
b) about 30 to about 90 weight percent vitamin E TPGS; and
c) from 0 to about 25 weight percent other therapeutically active compounds, wherein the summation of said weight percentages equals a total of 100 weight percent.

24. The composition according to claim 23 wherein said other therapeutically active compounds are non-antibiotic, non-steroidal, non-immunosuppressant, and non-monoclonal antibody compounds.

25. The composition according to claim 23 wherein said esters of gamma-tocopherol and said esters of gamma-tocotrienol include the succinate, polyethylene glycol succinate, acetate, nicotinate, and phosphate esters thereof.

26. The composition according to claim 23 wherein said gamma-homolog of vitamin E is gamma-tocopherol or an ester thereof.

27. An oral dosage form of the composition according to claim 23.


SEQUENCE LISTING (1 sequence)

SEQ ID NO: 1 - DNA, 22 bp, Artificial
  Transcription factor probe
agttgaggggactttcccaggc